(19) United States
(12) Patent Application Publication
(10) Pub. No.: US 2008/0145449 A1
(43) Pub. Date: Jun. 19, 2008

(54) Thiol reactive agents as a therapeutic modality

(75) Inventor: Jonathan S. Stamler, Chapel Hill, NC (US)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(73) Assignee: Duke University

(21) Appl. No.: 12/070,977

(22) Filed: Feb. 22, 2008

Related U.S. Application Data
(62) Division of application No. 10/677,752, filed on Oct. 3, 2003.
Application No. 10/677,752 is a continuation-in-part of application No. 10/608,120, filed on Jun. 30, 2003, now Pat. No. 6,964,984.
Application No. 10/608,120 is a continuation of application No. 10/280,085, filed on Oct. 25, 2002, now Pat. No. 6,627,602.
Application No. 10/280,085 is a continuation-in-part of application No. 09/986,807, filed on Nov. 13, 2001, now Pat. No. 6,472,390.

(51) Int. Cl. A61K 33/00; A61K 31/145; A61K 33/06; A61K 31/444; A61K 33/36; A61K 33/04; A61K 31/12; A61K 33/40; A61K 38/06; A61P 21/00; A61P 25/00

(52) U.S. Cl. 424/610; 514/508; 424/696; 514/332; 424/621; 424/703; 514/678; 424/616; 514/18

(57) ABSTRACT

A patient with a disease associated with a receptor having a cysteine residue is treated with a thiol reactive agent. The diseases include neurodegenerative diseases. Diseases characterized by skeletal muscle atrophy are also treated.

### CROSS-REFERENCE TO RELATED APPLICATIONS

This application is a divisional of U.S. application Ser. No. 10/677,752 filed Oct. 3, 2003, which is a continuation-in-part to U.S. application Ser. No. 10/608,120 filed Jun. 30, 2003, which in turn is a continuation of U.S. application Ser. No. 10/280,085, filed Oct. 25, 2002, now U.S. Pat. No. 6,627,602 which in turn is a continuation-in-part of U.S. application Ser. No. 09/986,807, filed Nov. 13, 2001, now U.S. Pat. No. 6,472,390.

### TECHNICAL FIELD

In one case, this invention is directed to prophylaxis or treatment of a patient with a disease associated with a protein having a cysteine residue. In other cases, the invention is directed to prophylaxis or treatment of a patient with a neurodegenerative or a patient with a disease characterized by skeletal muscle atrophy.

### BACKGROUND OF THE INVENTION

It is known that nitric oxide including all redox related forms, congers and donors (NO) regulates the function of most classes of protein by S-nitrosylation, that is, NO binds to or reacts with thiol residues to either inhibit or activate proteins. It is also known that S-nitrosylation can promote the formation of disulfides in the case of proteins containing redox sites comprised of vicinal thiols (i.e., to promote the formation of disulfides by S-nitrosylating at the vicinal thiols) or to inhibit formation of disulfides by S-nitrosylation at other (non-vicinal) sites, with the presence or absence of disulfides modulating the activity of the protein, for example, its reactivity with NO, its activity or its interaction with other proteins, and the effect thereof. Relying on the protein regulating function of NO, U.S. Pat. No. 6,472,390 claims a method for prophylaxis or treatment of a patient with a disease associated with a receptor having a cysteine residue or other cysteine containing protein to inhibit its function, or at risk therefor, comprising administering to said patient an NO donor that donates nitric oxide or a related redox species and provides bioactivity that is identified with nitric oxide. A potential disadvantage in administering NO donor is that such administration acutely lowers blood pressure and such blood pressure lowering may be counterindicated in respect to the disease being treated. In addition, NO can have other toxicities. A discovery in respect to U.S. Pat. No. 6,472,390 is that doses of NO donor which are insufficient to acutely lower mean arterial blood pressure or pulmonary artery pressure more than 10%, provide benefit.

Certain thiol reactive agents different from NO and NO donors are known to function the same as NO in some cases. For example, both NO and bisindolylmaleimide promote tumor necrosis factor induced apoptosis. Moreover, both NO and glutathione disulfide are known to activate the RyR gene to release calcium and enhance contractility and that an excess of both also inhibits contractility. Furthermore, both NO and mercurials are known to activate metaloproteinases to induce cell death on the one hand and protect endothelium on the other. Moreover, it is also known that reactive aldehydes and reactive nitrogen species react with thiols and that organisms share common defenses against the aldehydes and reactive nitrogen species. For example, it has been discovered by us that alcohol and aldehyde dehydrogenases protect against both aldehydes and S-nitrosoglutathione.

Moreover, thiol reactive agents are known to modify the reactivity of proteins toward NO. For example, arsenicals are known to cause disulfides to link or to cause formation of mixed disulfides, and diamides are known to catalyze disulfide formation whereas alkylators, e.g., maleimides, are known to block disulfide formation. Moreover, it is known from the case of ryanodine receptors, that proteins may contain redox modulatory sites, e.g., disulfides, and additionally single cysteines, that have entirely different reactivities toward different thiol reactive agents. Thus, some cysteines react better with some disulfides, some react better towards reactive oxygen species, some react better toward reactive nitrogen species, and others better toward other oxidants or alkylators. It is also known that the effects of NO may be critically dependent on the redox state of a redox modulating site, i.e., endogenous NO may react if the modulatory site is in the appropriate redox state. See Sun, J., et al, J. Biol. Chem. 276, 15625-15630 (2001) and Eu, J., et al, Cell 102, 499-509 (2001). Moreover, we have previously discovered that modification of single critical (function regulatory) cysteines in proteins by S-nitrosylation can have entirely different effects from modification of the same cysteines by oxidation by some thiol reactive agent whereas oxidations by other thiol reactive agents can produce similar effects, e.g., S—NO, sulfenic versus sulfinic versus mixed disulfides. See Kim, S., Cell 109, 383-396 (2002).

Moreover, certain thiol reactive agents different from NO donors have been used to treat certain diseases without knowledge of how they function. For example, arsenicals are known to treat leukemia. Gold compounds are known to treat asthma and rheumatoid arthritis. Nitroxyl anion/Angeli's salt has been administered to improve heart function. Thiol reactive agents have been shown to reduce sickling of hemoglobin. Bis-indolylmaleimide has been indicated to be active in treating some cancers. Except in the case of NO, those skilled in the art do not know what in a protein to target with thiol reactive agents or that different thiol reactive agents can have different effects.

Moreover, U.S. application Ser. No. 09/403,775, now U.S. Pat. No. 6,617,355, discloses administration of inhibitors of S-nitrosothiol breakdown including inhibitors of enzymes and non-enzymatic proteins containing thiol groups, including N-ethylmaleimide, to treat asthma.

Heretofore it has not been appreciated that thiol reactive agents different from NO and NO donors can be administered to target the same sites in proteins as NO and NO donors target, to provide prophylaxis or treatment benefit with not as much potential of causing blood pressure to lower as do NO and NO donors and without the toxicity inherent in NO donors, i.e., without the reactions of NO with oxygen to produce toxic NO, and related mutagenic products.

Moreover, it has been discovered herein that there are certain critical sites in proteins that mediate regulation of protein function and that oxidation or covalent modification of some sites by thiol reactive agents allows both endogenous NO and exogenous NO to work better and that these sites can be targeted by very different reactivities of different thiol reactive agents. Moreover, it has been discovered herein that there are multiple different classes of thiol in proteins characterized by different reactivities to a reagent and that different thiols that have a common reactivity to a thiol reactive agent, when modified, elicit different effects. Turning now to the different classes of thiols in proteins, in some cases, thiol reactivity is linked to other thiols and in other cases not linked to other thiols. Knowing the above allows one skilled in the art to target thiols to obtain desired effects. It cannot be predicted that if a particular effect is obtained with some thiol reactive agent, that the same effect will be obtained with a different thiol reactive agent. However, it can be predicted that one can obtain a desired effect with thiol reactive agent different from NO or an NO donor if that effect was obtained by administration of NO or an NO donor.

It has also been discovered herein that allosteric thiols exist across all classes of proteins to provide opportunity for modification to provide therapeutic effect and that modulation of allosteric sites of proteins has subtle (gentle) effects, i.e., the protein is sensitized to inhibition or to activation. This is important because more excessive effects can kill the protein and the patient.

In summary, it has been discovered herein that thiol reactive agents different from NO and NO donors can be administered to target the same sites in proteins as NO and NO donors target, that there are different classes of thiol reactive sites in proteins (e.g., interaction with disulfide-forming-single cysteine-allosteric sites), that the function of different thiol reactive sites in a protein may be linked, that modification of a single site (e.g., SNO, SOH, SSG or SX where X is an alkylator or protein) with a different reagent can produce a different effect and that there are specific sites in proteins that can be targeted by thiol reactive agents different from NO and NO donors for therapeutic effect and that libraries can be used to find thiol reactive agents for specific targets.

It has also been discovered herein that thiol reactive agents can be reacted with protein thiol to regulate protein-protein interactions and thus the functional consequences thereof.

Moreover, it has been discovered herein that thiol reactive agents, including NO and NO donors, are useful to treat patients with neurodegenerative diseases or diseases characterized by skeletal muscle atrophy.

### SUMMARY OF THE INVENTION

In one embodiment, denoted the first embodiment, the invention herein is directed to a method for prophylaxis or treatment of a patient with a disease associated with a protein having a cysteine residue that is modified by a thiol reactive agent to modulate its function or to inhibit or promote its function, or at risk therefor comprising administering to said patient a therapeutically effective amount of said thiol reactive agent, with the proviso that the thiol reactive agent is not NO or an NO donor. Excluded from this embodiment is the use of gold compounds and N-ethylmaleimide to treat asthma, the use of gold compounds to treat rheumatoid arthritis, the use of arsenicals to treat leukemia, the use of bis-indolylmaleimide to treat those cases of cancers where it is know for use and the use of nitroxyl anion/Angeli's salt to improve heart function, and in other cases where thiol reactive agents may have been used to treat disorders without knowledge of how they are functioning.

In another embodiment denoted the second embodiment, the invention is directed at a method of prophylaxis, i.e., to induce a protective response, against stroke, heart attack or ischemic disorder comprising administering to a patient at risk for stroke, heart attack or ischemic disorder, a therapeutically effective amount of a thiol reactive agent different from NO and NO donors.

In another embodiment denoted the third embodiment, the invention is directed to treating a patient with a fungal disorder, comprising administering to the patient a therapeutically effective amount of thiol reactive agent different from NO or an NO donor which reacts with a function regulating cysteine in fungal ABC transporter or kinase which is not present in mammalian ABC transporter or kinase, to kill the fungus.

In still another embodiment denoted the fourth embodiment, the invention is directed to a method of treating diseases associated with protein-protein interaction where at least one of the proteins has more than one function regulating cysteine in an allosteric site, comprising administering to a patient having such disease, a therapeutically effective amount of a thiol reactive agent different from NO or an NO donor which is selective or effective for one of the function regulating cysteines.

In another embodiment, denoted the fifth embodiment, the invention is directed at a method for the prophylaxis or treatment of a patient with a neurodegenerative disease associated with a protein having a cysteine residue that is modified by a thiol reactive agent including NO and NO donors, to inhibit its function, or at risk therefor, comprising administering to said patient a therapeutically effective amount of said thiol reactive agent, provided that when the agent administered is NO or an NO donor is administered, it is administered in an amount which provides a submicromolar concentration of the NO or NO donor in the patient's blood.

In still another embodiment, denoted the sixth embodiment, the invention is directed at a method for prophylaxis or treatment of a patient with a disease characterized by skeletal muscle atrophy, or at risk therefor, comprising administering to said patient a therapeutically effective amount of a thiol reactive agent including NO and NO donors, thereby to stimulate growth of skeletal muscle.

The term “NO” is used herein to include nitric oxide gas related redox species and oxidation states and oxidized derivatives thereof.

The term “NO donor” is used herein to mean a compound that donates nitric oxide or a related redox species and more generally provides nitric oxide bioactivity, that is activity which is identified with nitric oxide, e.g., vasorelaxation or stimulation or inhibition of a receptor protein, e.g., ras protein, adrenergic receptor, NFκB.

The term “thiol reactive agent” is used herein to mean compound that binds to or reacts with thiol residue of a protein (excluding active site cysteines in enzymes) including receptors and other proteins to either inhibit or activate the protein.

### DETAILED DESCRIPTION

We turn now to the first embodiment of the invention, which is directed to a method for prophylaxis or treatment of a patient with a disease associated with a protein having a cysteine residue, including receptors and other proteins, that is modified by a thiol reactive agent to modulate its function or to inhibit or promote its function, or at risk therefor, comprising administering to said patient a therapeutically effective amount of said thiol reactive agent, with the proviso that the thiol reactive agent is not NO or an NO donor. The protein having a cysteine residue is a protein where allosteric cysteines are regulatory, i.e., cysteines that regulate function independent of active site and modification of the cysteine changes the function of the protein. The proteins contain one or more thiols or classes of thiols.

The term “disease associated with a protein having a cysteine residue” is used herein to mean a disease in which a thiol containing protein is dysfunctional or in which the modification of a protein can have a salutory effect, e.g., modulation of the NMDA receptor at the redox site to treat Alzheimer's disease or modulation of the β-adrenergic receptor or an associated protein where the receptor interacts to treat heart disease such as heart failure or lung disease such as asthma.

The term “modulate its function” as distinct from inhibiting or promoting its function, is used herein to mean to alter the activity of the protein or of other inhibiting or promoting agent or molecule.

The proteins referred to in the general description of the first embodiment include, for example, serotonin receptors, adrenergic receptors, blood cell membrane receptors, μ-opioid receptors, G-protein coupled receptors, receptors that are not G-protein coupled, G-proteins, metabolic proteins, receptors that are structural or adaptor proteins, receptors that are membrane proteins, receptors that are intracellular proteins, kinases, receptors that are phosphatases, receptors that are cysteine proteins where the treatment would affect an allosteric cysteine, receptors that are cyclins, ion channel proteins, receptors that are transcription factors and receptors that are respiratory proteins.

Diseases associated with serotonin receptors, treatable in the first embodiment herein, include, for example, depression, stress and/or anxiety, and atherosclerosis.

Diseases associated with α-adrenergic receptors, treatable in the first embodiment herein, include, for example, benign prostatic hypertrophy and urinary incontinence.

Diseases associated with β-adrenergic receptors, treatable in the first embodiment herein, include, for example, systemic hypertension, pulmonary hypertension, coronary artery disease, right or left heart failure, cases where a patient is on a left ventricular heart assist device awaiting heart transplant, cases where a patient who has had heart surgery and cannot be disconnected from a heart pump without loss of heart function, cases where a patient is undergoing surgery who is at risk for a cardiac event, and stroke.

Diseases associated with blood cell membrane receptors, that are treatable in the first embodiment herein, include, for example, ischemic disorders, sickle cell disease and thalassemias.

Diseases associated with μ-opioid receptors, treatable in the first embodiment herein, include, for example, cases where a patient is being treated with an opiate because of severe pain because of surgery, cancer or accidental injury and cases where a patient is addicted to an opiate.

Diseases associated with G-protein coupled receptors, that are treatable in the first embodiment herein, include, for example, heart failure, infection or asthma.

Diseases associated with receptors that are not G-protein coupled, that are treatable in the first embodiment herein, include, for example, cancer of many types.

Diseases associated with G-proteins, that are treatable in the first embodiment herein, include, for example, cancers, for example, lung cancers and gastrointestinal cancers.

Diseases associated with metabolic proteins, that are treatable in the first embodiment herein, include, for example, sickle cell disease and diabetes.

Diseases associated with receptors that are structural or adaptor proteins, that are treatable in the first embodiment herein, include, for example, infection, Alzheimer's disease, Parkinson's disease, Huntington's disease, post-CABG dementia, neuropathic pain, ALS, depression, AIDS dementia and muscular sclerosis.

Diseases associated with receptors that are membrane proteins that are treatable in the first embodiment herein, include, for example, viral infections, hypertension, cystic fibrosis and myasthenia gravis.

Diseases associated with receptors that are intracellular proteins, that are treatable in the first embodiment herein, include, for example, sickle cell disease, muscular dystrophy, Alzheimer's disease and Parkinson's disease.

Diseases associated with proteins that are kinases, that are treatable in the first embodiment herein, include, for example, asthma, heart failure and cancers, for example leukemias and lung cancers.

Diseases associated with receptors that are phosphatases, that are treatable in the first embodiment herein, include, for example, heart failure, Alzheimer's disease and cancers, for example, melanoma and breast cancer.

Diseases associated with receptors that are cysteine proteins where the treatment would affect an allosteric cysteine, that are treatable in the first embodiment herein, include, for example, all degenerative disorders, including for example, Huntington's disease, Alzheimer's disease, atherosclerosis, heart failure, AIDS dementia, amyotrophic lateral sclerosis (ALS) and muscular sclerosis.

Diseases associated with receptors that cyclins, that are treatable in the first embodiment herein, include, for example, cancers and diseases where stem cell therapy is applicable.

Diseases associated with ion channel proteins, that are treatable in the first embodiment herein, include, for example, arrhythmias, epilepsy, stroke and myopathy.

Diseases associated with receptors that are transcription factors, that are treatable in the first embodiment herein, include, for example, asthma, viral infections and rheumatoid arthritis.

Diseases associated with receptors that are respiratory proteins, that are treatable in the first embodiment herein, include, for example, Franconi's anemia, Leigh's encephalopathy and thalassemias.

The proteins having a cysteine residue that are reacted in the treatments in the first embodiment herein, include, for example, those in endothelial cells, cardiac cells, epithelial cells, nerve cells, neutrophils, leukocytes, fibroblasts, platelets where the receptor is not an adenosine diphosphate (ADP) receptor, bone marrow cells, skeletal muscle cells and stem cells or stem cell lineage related cells.

Diseases associated with receptors in endothelial cells, treatable in the first embodiment herein, include, for example, inflammatory diseases, for example, atherosclerosis, ischemia, reperfusion injury, diseases benefiting from cardiac preconditioning, diabetes and peripheral vascular disease.

Diseases associated with receptors in cardiac cells, treatable in the first embodiment herein, include, for example, heart failure and myocardial hypertrophy.

Diseases associated with receptors in epithelial cells, treatable in the first embodiment herein, include, for example, asthma, chronic obstructive pulmonary disease (COPD) and cystic fibrosis.

Diseases associated with receptors in nerve cells, treatable in the first embodiment herein, include, for example, stroke, neuropathic pain, glaucoma and neurodegenerative disorders, e.g., Huntington's disease, Alzheimer's disease, Parkinson's disease and amyotrophic lateral sclerosis.

Diseases associated with receptors in neutrophils, that are treatable in the first embodiment herein, include, for example, inflammatory conditions, for example, sepsis, asthma, rheumatoid arthritis, atherosclerosis, diabetes and peripheral vascular disease.

Diseases associated with receptors in leukocytes, treatable in the first embodiment herein, include, for example, multiple sclerosis and rheumatoid arthritis.

Diseases associated with receptors in fibroblasts, that are treatable in the first embodiment herein, include, for example, pulmonary fibrosis.

Diseases associated with receptors in platelets that are not ADP receptors, that are treatable in the first embodiment herein, include, for example, atrial fibrillation (AF), thrombosis, disseminated intravascular coagulation and idiopathic thrombocytopenic purpura (ITP).

Diseases associated with receptors in bone marrow cells, that are treatable in the first embodiment herein, include, for example, anemia, leukemia, polycythemia and aplasia.

Diseases associated with receptors in skeletal muscle cells, that are treatable in the first embodiment herein, include, for example, muscular dystrophy, COPD, respiratory failure and heart attack.

Diseases associated with receptors in stem cells or stem cell lineage related cells, that are treatable in the first embodiment herein, include, for example, stroke, atherosclerosis, anemia, leukopenia, thrombocytopenia or a deficiency in any cell of any tissue.

The cysteine containing proteins other than receptors in the classical sense, that are referred to in the general description of the first embodiment herein, include, for example, G-proteins and G-proteins receptor kinases (GRKs) and also include, for example, NFκB, API, ras, Na⁺ channels, Ca²⁺ channels, K⁺ channels, and prion proteins. (See Stamler, J. S., Cell, 2001.) Diseases not mentioned above associated with these proteins that are not receptors that are treatable by the first embodiment of the invention herein, include, for example, prion related diseases, e.g., Creutzfeldt-Jacob disease, kuru and mad cow disease, and malignant hyperthermia.

General classes of diseases whose prophylaxis and treatment are embraced by the first embodiment herein, include, inflammatory conditions except for some cases of asthma (asthma has been treated with some compounds that are thiol reactive agents but are not NO donors before the invention herein) and diseases or conditions characterized by pathologically proliferating cells. The inflammatory conditions include, for example, asthma, rheumatoid arthritis, atherosclerosis, ischemic reperfusion injury, diabetes and peripheral vascular disease. The diseases or conditions characterized by pathologically proliferating cells include, for example, restenosis, benign prostatic hypertrophy and cancers, including, for example, Hodgkin's disease, small cell lung cancers, cancer of the breast and testicular and prostate cancer.

In one variation of the first embodiment asthma is excluded from the diseases treated.

Thiol reactive agents for use in the first embodiment herein, include, for example, avicins, arsenicals, e.g., arsenic trioxide, for use other than for treating leukemia, thiol arsenides, selenium compounds including selenite (SeO₃⁻) and GS₂—As—Se, gold compounds for use other than for treating asthma and rheumatoid arthritis, maleimides other than to treat asthma, formamides, nicotinamide adenine dinucleotide, hydrogen peroxide (which activates ryanodine receptors to cause reaction with thiols), hydrogen sulfide (e.g., in a concentration of 0.1 to 10 or 100 ppm in nitrogen or other inert gas) thiol reactive aldehydes, e.g., betaine aldehyde, formaldehyde and pyridoxyl phosphate, quinones, e.g., napthoquinone, menadione and Vitamin K, sulfides, e.g., glutathione disulfide, lactones, e.g., β-propiolactone, penicillin and lipoic acid disulfide, S-sulfo derivatives, e.g., RSOR′, and epoxides. Preferred treating agents include sulforaphane, potassium terricyanide, 2,2′-dipyridyldisulfide, 4,4′-dipyridyldisulfide, selenite, arsenic trioxide, hydrogen peroxide, (2-hydroxybenzylidine acetone), (4-hydroxy benzylidine acetone), [bis(2-4-hydroxybenzylidine acetone)], antabuse, and memantine-L-2-pyridyldithiol propionamide. In one variation of the invention, penicillin and/or hydrogen sulfide are excluded as a treating agent. The ability to establish the effect of different thiol reactive agents is well established. See Kim et al, Cell 109, 383-396 (2002); Kim et al, Neuron 24, 461-469 (1999); and Xu et al, Science 79, 234-237 (1998). In each of these papers, different classes of thiol reactive agents are shown to have profoundly different effects on the function of distinct classes of proteins to elicit varying effects and on the transcription of different genes, in one case genes that protect bacterial cells, in another case a protein that regulates neuronal plasticity and in still another case a protein that controls force in the heart.

We turn now to selection of thiol reactive agent for use to treat a particular disease. The association between proteins and many diseases is set forth above. Moreover, it is known that inhibition of kinases is a treatment for some cancers and it is anticipated that inhibition of kinases may broadly treat other diseases and infections (in the case of a bacterial kinase, for example); and it is known that inhibition of ion channels is a treatment for heart failure and inhibition of GRKs is a treatment for asthma. Thus, the proteins for targeting are by-in-large determined. In some cases the cysteines for targeting are known. For example, there is a cysteine in guanylate cyclase that is known in the literature to be a site for pharmaceutical interest; this provides a pertinent cysteine herein. Moreover, pertinent cysteines have and may be identified through S-nitrosylation. And even if a particular cysteine has not been determined as being relevant, screening can be carried out. The point is to screen for thiol reactive agents that react with cysteines in a protein whether a particular cysteine is known to be of interest or not and to screen for reaction with particular cysteine in a protein if it is known to be of interest. Screening is readily carried out. Classes of compounds that react with proteins are large and well known. See, for example, Jocelyn, Biochemistry of the SH Group, Academic Press, London 1992. Moreover, there are commercially available libraries of compounds that react with cysteines. These compounds and those libraries can be studied for effect. In addition, critical NO sensitive or redox sensitive motifs can be readily identified; see Stamler, J. S., et al, Neuron 18, 691-696 (1997) and Fomenko and Gladyshev, Biochemistry 42, 11214-11225 (2003). In summary, small molecules known to react with cysteines can be screened for effect in the case of a particular protein associated with a disease or a particular cysteine of interest in that protein. As long as the compound is determined to react with a cysteine, it is of interest regardless of its potency. Compounds with low effect or low specificity can be modified to obtain greater effect and/or specificity. In many cases, greater specificity can be achieved simply by changing the solubility or lipophilicity of the thiol reactive agent. For example, a thiol reactive agent can be made cell impermeable, e.g., by attaching a charged residue, where targeting of extracellular cysteine is desirable. For example, in the case of stroke and atherosclerosis, it is known that activation of receptors, e.g., NMDA receptors, EGF receptors and PDGF receptors, is involved in providing symptoms; thiol targeting of these receptors will ameliorate the symptoms. Thiol reactive agents of the class bimanes, qBBr and mBBr are known examples of cell impermeable and cell permeable thiol reactive agents respectively and have been used experimentally for other purposes. So the precedent is well established making thiol reactive agents that enter or do not enter cells for experimental purposes. The present invention adopts this technique for therapeutic purposes.

It is not heretofore been appreciated that cysteines are ubiquitous in all classes of proteins that may be targeted by thiol reactive agents to modify the effect of the proteins.

In U.S. Pat. No. 6,472,390 NO donors are used to modify cysteines in proteins. This invention extends this modification to thiol reactive agents besides NO and NO donors.

The therapeutically effective amount is an amount that ameliorates a symptom or symptoms of the condition being treated or in the case of prophylaxis an amount that prevents symptom(s) from occurring or causes the symptom(s) which occur to be less in intensity than those that would occur without the administration of the first embodiment of the invention. In general, administering a therapeutically effective amount where the thiol reactive agent is administered systemically, involves administration in an amount to achieve a concentration of thiol reactive agent in the blood of 100 picomolar to 100 micromolar with the specific dosage depending on the drug administered and the disease treated or at risk for. Where the thiol reactive agent is administered to provide specificity, e.g., locally, the dosage ranges from 1 nanomolar to 1 millimolar or 1 μg to 1,000 mg/day, with specific dosage depending on the drug administered and the disease treated or at risk for.

We turn now to the routes and methods of administration for the first embodiment, whereby the thiol reactive agent reaches and reacts with the cysteine residue of the protein. In some cases, systemic administration, e.g., intravenous administration or oral administration, is appropriate, e.g., if the protein is in blood vessels or if the patient is dying, e.g., has septic shock. In cases where administration is preferably with specificity via-a-vis some receptor, the thiol reactive agent is preferably attached to a receptor agonist or antagonist, e.g., to a receptor antagonist when the receptor is an enzyme. In cases where specificity can be provided by local administration (e.g. by inhalation into the lungs), local administration is appropriate.

We turn now to the second embodiment which is directed to a method of prophylaxis, i.e., to induce a protective response, against stroke, heart attack or ischemic disorder, comprising administering to patient at risk for stroke, heart attack, or ischemic disorder, a therapeutically effective amount of a thiol reactive agent different from NO and NO donors. This method includes instituting the phenomenon of preconditioning and includes activation of HIF (hypoxia inducible factor) which is a transcription factor that contains a function regulatory thiol, to protect cells from lack of oxygen, and to provide induction of genes to provide against oxidative and nitrosative stress.

The thiol reactive agents and routes and methods of administration for the second embodiment herein are the same as those for the first embodiment herein. Determination of appropriate thiol reactive agent can be effected by the method for selection of thiol reactive agent set forth in the first embodiment. The therapeutically effective amount is an amount which prevents symptom(s) from occurring or causes the symptom(s) which occur to be less in intensity than those that would occur without the administration of the second embodiment of the invention and in general ranges from 1 nanomolar to 1 millimolar (concentration in blood) or 1 μg to 1,000 mg/day, with specific dosage depending on the drug administered and the condition at risk for.

We turn now to the third embodiment of the invention herein which is directed to treating a patient with a fungal disorder, comprising administering to the patient a therapeutically effective amount of thiol reactive agent different from NO or an NO donor which reacts with a function regulatory cysteine in fungal ABC transporter or kinase which is not present in mammalian ABC transporter kinase, to kill the fungus. Determination of thiol reactive agent can be from among those, e.g., as set forth in the first embodiment where the proteins are fungal ABC transporters or kinases and mammalian ABC transporters or kinases. Dosage and method of administration are as set forth in the first embodiment. The method of the third embodiment is specifically exemplified in Example XXXVII hereinafter.

We turn now to the fourth embodiment of the invention herein which is directed to a method of treating diseases associated with protein-protein interactions where at least one of the proteins has more than one function regulating cysteine in an allosteric site, comprising administering to a patient having such disease, a therapeutically effective amount of a thiol reactive agent different from NO or an NO donor which is selective or effective for one of the function regulating cysteines. The basis for this is that when there are two cysteines in a protein, the reactivity of each to NO is quite different. When there is a difference in activity with NO or an NO donor, there is a difference in activity with other thiol reactive agents.

Diseases treated in the fourth embodiment include, or example, heart failure, asthma and stroke.

In general, the thiol reactive agents for the fourth embodiment herein are the same as for the first embodiment herein with a specific thiol reactive agent being determined as being selective for the one cysteine. The establishing of appropriate thiol reactive agent is by the same method as set forth in the first embodiment.

The methods and routes of administration for the fourth embodiment herein are the same as for the first embodiment herein.

A therapeutically effective amount for the fourth embodiment herein is an amount which ameliorates a symptom or symptoms of the condition being treated and in general the dosage ranges from 1 nanomolar to 1 millimolar concentration in blood or 1 μg to 1,000 mg/day with variation according to particular treating agent and the condition treated.

The method of the fourth embodiment is specificity exemplified in Examples XLVII and XLVIII hereinafter.

We turn now to the fifth embodiment of the invention herein, which is directed at a method for the prophylaxis or treatment of a patient with a neurodegenerative disease associated with a protein having a cysteine residue that is modified by a thiol reactive agent to inhibit its function, or at risk therefor, comprising administering to said patient a therapeutically effective amount of a thiol reactive agent including NO and NO donors, provided that when the agent is NO or an NO donor, it is administered in an amount which provides a submicromolar concentration of the NO or NO donor in the patient's blood.

The neurodegenerative diseases associated with a protein having a cysteine residue for the fifth embodiment herein, include, for example, Huntington's disease, Alzheimer's disease, Parkinson's disease and amyotrophic lateral sclerosis.

The thiol reactive treating agents for the fifth embodiment include not only the thiol reactive agents of the first embodiment, but also additionally nitric oxide and related redox species and oxidation states and oxidized derivatives thereof as well as NO donors. NO donors for use in the fifth embodiment include those listed in U.S. Pat. No. 6,472,390, the whole of which is included herein by reference. Preferred NO donors for use in the fifth embodiment include ethyl nitrite, S-nitrosoglutathione, nitrosoallopurinol and Angeli's salt. The thiol reactive agent is selected by the method of selecting set forth in the first embodiment.

The therapeutically effective amount for the fifth embodiment, is an amount that ameliorates a symptom or symptoms of the neurodegenerative disease being treated, or in the case of prophylaxis, an amount that prevents symptom(s) from occurring or causes the symptom(s) which occur to be less in intensity than those that would occur without the administration of the fifth embodiment of the invention. In general, administering a therapeutically effective amount involves administering a dosage ranging from 1 nanomolar to 1 millimolar concentration in blood or 1 μg to 1,000 mg/day, with the specific dosage depending on the drug administered and the disease treated or at risk for. When NO or an NO donor is the thiol reactive agent, it is administered in a dosage to provide a nanomolar to submicromolar (e.g., 1 nanomolar to 100 nanomolar) concentration of NO or NO donor in the patient's blood. This allows selectivity for one thiol in a protein and distinguishes the use where millimolar concentrations of NO have been used to activate ryanodine receptors in vitro.

Administration for the fifth embodiment, can be, or example, intrathecally, intraventricularly, or intravenously in disease states where the blood-brain barrier is not intact.

We turn now to the sixth embodiment of the invention herein, which is directed at a method for prophylaxis or treatment of a patient with a disease characterized by skeletal muscle atrophy, or at risk therefor, comprising administering to said patient a therapeutically effective amount of a thiol reactive agent including NO and NO donors, thereby to stimulate growth of skeletal muscle.

Diseases for the sixth embodiment include muscular dystrophy, COPD and respiratory failure.

The thiol reactive agents for the sixth embodiment are the same as those for the fifth embodiment. Selection of appropriate thiol reactive agent is by the selection method set forth in the first embodiment.

Dosage for the thiol reactive agents for the sixth embodiment is an amount that stimulates growth of the skeletal muscle involved as measured by ryanodine receptor binding of ryanodine, muscle specific protein expression, NFAT activity and fiber type, and ranges from 1 nanomolar to 1 millimolar concentration in blood or 1 μg to 1, 000 mg/day, with the specific dosage depending on the drug administered and the disease treated or at risk for.

Routes of administration for the sixth embodiment are, for example, oral, intravenous, topical and inhaled.

The invention is illustrated by the following examples:

### EXAMPLE 1

A 60-year-old woman with depression, unresponsive to medical therapy, receives 40 mg PO QID of sulforaphane for 3 weeks and symptoms of depression improve.

### EXAMPLE II

A 26-year-old white female with severe anxiety disorder receives 1 mg P.O. BID of potassium terricyanide with relief of anxiety in two days.

### EXAMPLE III

A 72-year-old white male with severe atherosclerosis, unresponsive to medical therapy including nitrates, beta blockers and aspirin, begins daily therapy with selenite, 1 mg P.O., QD Whereas prior to initiation of therapy the patient experienced angina with two-block exertion, he was able to increase his exercise regimen to 4 blocks without pain.

### EXAMPLES IV AND V

A 70-year-old black male with benign prostatic hypertrophy and severe urinary frequency (Q 2 hrs) begins therapy with 4,4′-dipyridyl disulfide, 4 ml P.O. BID with improvement in symptoms; the patient's urinary frequency decreases to every 4 hours.

### EXAMPLE VI

A 27-year-old white female with frequent asthma exacerbations is admitted to the intensive care unit and intubated. She receives full medical treatment, including steroids, beta agonists and theophylline with little response. The patient is begun on inhaled selenite, 1 millimolar in 2 cc normal saline, 0.4 hours, nebulized. She is extubated within 48 hours.

### EXAMPLE VII

A 46 year old black male with a blood pressure of 200/100 mm Hg enters the emergency room. He is begun on captopril and hydrochlorothiazide and his blood pressure drops to 180/110 mm Hg. The patient is subsequently begun on 4,4′-dipyridyl disulfide, 25 mg/kg, and the blood pressure improves over 24 hours to 160/100 mm Hg.

### EXAMPLE VIII

A 28-year-old white female with primary pulmonary hypertension and pulmonary pressures of 80/40 mm Hg enters the hospital complaining of shortness of breath. She is begun on inhaled arsenic trioxide (2 cc of a 100 micromolar solution), inhaled Q.4 hours, and her pulmonary pressure falls to 60 mm Hg. The PO₂ improves from 85 to 90.

### EXAMPLE IX

A 70-year-old with unstable angina presents at the emergency room and is begun on oral arsenic trioxide, 10 mg P.O. QD. The patient has a heart attack much smaller than expected based on region of risk.

### EXAMPLE X

A 30-year-old white male with dilated cardiomopathy and severe right heart failure receives an IV infusion of glutathione disulfide at 25 mg/kg QID. Right ventricular function, accessed by echocardiography, improves, and pulmonary pressures fall from a mean P of 30 to 25. The patient is then begun on oral arsenic trioxide, 10 mg P.O., QD. The patient's peripheral edema improves over 3 days.

### EXAMPLES XI AND XII

A 65-year-old with an ischemic cardiomopathy and a left ventricular ejection fraction of 15% is placed on a left ventricular heart assist device and awaits transplantation. The patient experiences severe shortness of breath and is begun on 1 nmol/kg/min intravenous hydrogen peroxide and the symptoms of shortness of breath improve. The ejection fraction improves over 24 hours to 20%. The patient is then begun on selenite, 10 mg, P.O., QD, with improvement in ejection fraction to 30% over 2 weeks.

### EXAMPLE XIII

A 62-year-old white male with an ischemic cardiomopathy undergoes coronary artery bypass surgery and cannot be disconnected from a bypass pump. Intravenous arsenic trioxide is administered to give a final blood concentration of 1 μm, and the patient is disconnected from bypass successfully.

### EXAMPLE XIV

A 50-year old with stroke is admitted and is given inhaled H₂S at 10 ppm with improvement in state of consciousness after 12 hours.

### EXAMPLE XV

A 72-year-old white male with an acute left hemispheric stroke is admitted to the emergency room. The patient is given memantine-L-2-pyridyl dithiol propionamide, 20 mg P.O., QD. The blood pressure remains stable at 180 mm Hg and the patient's mentation improves. The symptoms of stroke improve over the ensuing two weeks.

### EXAMPLE XVI

A 30-year old black female with sickle cell disease presents with a painful crisis. Antabuse is given at 0.5 g/day orally and the symptoms of crisis resolve over two hours. The patient is then begun on selenite, 1 mg/day orally. There is a decrease in frequency of painful crisis from three times per year to once per year.

### EXAMPLE XVII

The same as Example XVI except that the patient has an ischemic disorder, namely restenosis. The patient is given 10 mg/day arsenic trioxide orally with regression of restenosis.

### EXAMPLE XVIII

The same as Example XVII except that the patient has thalassemia and symptoms lessen.

### EXAMPLE XIX

A patient presents with severe neuropathic pain secondary to diabetes, unresponsive to all therapy. The patient is begun on codeine and oral 2,2′-dipyidyl disulfide, 4 mg. P.O. QID, and the symptoms of pain improve over 2 weeks.

### EXAMPLE XX

The same patient presented in Example XIX develops an opiate addiction. The initiation of 4,4′-dipyridyl disulfide allows the patient to taper off opiates over 1 week without serious side effects.

### EXAMPLE XXI

A 60-year-old smoker presents with a right upper lobe mass. The patient is begun on [bis(2-4-hydroxybenzylidene)acetone] in combination with chemotherapy. The patient's cancer shrinks by objective CT criteria after 3 weeks of therapy. The patient then goes to the operating room and has the tumor removed.

### EXAMPLE XXII

A 40-year-old white female with gastrointestinal cancer and severe abdominal pain begins oral therapy with 40 mg P.O. QID of selenite. The symptoms of pain diminish over 48 hours.

### EXAMPLE XXIII

A 26-year-old with severe respiratory distress secondary to influenza infection is begun on inhaled hydrogen peroxide, 100 parts per million. Over the following two days her PO₂ improves from 60 to 90.

### EXAMPLE XXIV

A 30-year-old white female with severe pseudomonas pneumonia in the setting of cystic fibrosis is begun on inhaled arsenic trioxide, 1 millimolar 2 cc solution, Q 4 hrs, and the patient's clinical status as measured by PO₂ increases from 50 to 70, and sterilization of airway aspirates improve significantly over 48 hours. The pneumonia resolves.

### EXAMPLE XXV

A 4-year-old with myasthemia gravis is begun on oxidized glutathione, 600 mg P.O. QID. The patient's ptosis improves over a week.

### EXAMPLE XXVI

A patient with severe muscular dystrophy receives an infusion of oxidized glutathione, 200 mg/kg/min over 48 hours. The patient's peripheral weakness and diaphragmatic weakness improve. The patient, nevertheless, has some difficulty getting to the bathroom without assistance and is begun on 4,4′-dipyridyl disulfide, 2 mg P.O. QID with additional gain in strength.

### EXAMPLE XXVII

A 72-year old white female with Alzheimer's disease is begun on 4 mg P.O. QID of selenite. The patient's cognition improves over 2 months. A second patient with Alzheimer's disease is begun on 4,4′-dipyridyl disulfide, 10 mg P.O. QID. The patient's cognition, which had been deteriorating, stabilizes over 6 months.

### EXAMPLE XXVIII

A 62-year-old with Parkinson's disease receives 4 mg P.O. QID of Angeli's salt and the patient's tremor decreases. A second patient, 60 years of age, complaining of severe bradykinesia, begins bis(2-hydroxybenzylidene acetone), 10 mg P.O. QD, and the movement of his arms and expression improve.

### EXAMPLE XXIX

A 27-year-old with leukemia is begun on selenite, 10 mg, P.O. QID, in combination with his standard chemotherapeutic regimen. The patient enters a full remission.

### EXAMPLE XXX

A 40-year-old female, who works as a gardener, presents with invasive melanoma. Before excision, she receives topical 4,4′-dipyridyl disulfide solution, 2 cc of a millimolar stock applied 4 times/day. The size of the tumor decreases from 1 centimeter to 0.8, and excision is then performed.

### EXAMPLE XXXI

A 32-year-old white female with breast cancer receives oral therapy with 4,4′-dipyridyl disulfide, 10 mg P.O. QID, and the size of the breast mass decreases from 3 centimeters to 2.5 centimeters. A surgical excision is then performed.

### EXAMPLE XXXII

A 42-year old Huntington's disease patient presents with uncontrolled chorea and is begun on oral GSNO, 40 mg P.O. QID, with temporizing of the symptoms. In addition, the patient is begun on 4,4′-dipyridyl disulfide, 3 mg P.O. QID, which further attenuates the patient's symptoms.

### EXAMPLE XXXIII

A 40-year-old with AIDS dementia is begun on 40 mg P.O. QD of 4,4′-dipyridyl disulfide and 40 mg P.O. QID of 2-hydroxybenzylidene acetone, which stabilizes the patient's dementia and decreases viral load.

### EXAMPLE XXXIV

A 6-year-old with ALS is treated with 40 mg P.O. QID of isosorbide dinitrate and weakness improves, but progressive degeneration continues at a slow rate. The patient is then treated with intravenous memantine-N-ethyl maleimide 20 mg/day, which stops the deterioration.

### EXAMPLE XXXV

A 26-year old white female with multiple sclerosis is given 5 mg/day arsenic trioxide. An improvement in ability to walk is noticed.

### EXAMPLE XXXVI

A 40-year-old with incessant ventricular tachycardia receives intravenous infusion of Angeli's salt at 10 nmol/kg/min, which stops the arrhythmia.

### EXAMPLE XXXVII

A 60-year old with epilepsy is given memantine-L-2-pyridyl dithiol propionamide, 20 mg, P.O. QD. The frequency of seizures decreases.

### EXAMPLE XXXVIII

A 28-year old immunocomprised patient develops cryptococcus and is treated with arsenic trioxide, 40 mg P.O. QD for two weeks, with improvement. The patient also receives standard antifungal therapy but effects are seen above and beyond those normally realized with standard therapy.

### EXAMPLE XXXIX

A 40-year-old female with rheumatoid arthritis receives an injection into her knee joint of 4,4′-dipyridyl disulfide (2 cc of a 100 micromolar solution). The patient notes decreases in pain and swelling over the following two days.

### EXAMPLE XL

A 40-year-old with Fanconi's anemia receives 40 mg of selenite P.O. QD for 2 weeks with an improvement in the anemia.

### EXAMPLE XLI

A 26-year-old with Leigh's encephalopathy receives oral arsenic trioxide, 10 mg/day, with improvement in encephalopathic changes over 2 days.

### EXAMPLE XLII

A 65-year-old with a ventricular arrhythmia induced by angioplasty to the artery receives an IV infusion of GS₂—As—Se at 10 nmol/kg/min. Angioplasty is then performed on a second vessel without ventricular arrhythmia. Myocardial stunning is not seen.

### EXAMPLE XLIII

A 40-year-old with ejection fraction of 30% and a 95% proximal LAD occlusion receives an intravenous infusion of GS₂—As—Se at 10 nmol/kg/min for 24 hours. The patient has an infarct, which is much smaller than had been anticipated. Ejection fraction improves by 5-10% over the following day.

### EXAMPLE XLIV

A 40-year-old with severe diabetes and pain on walking 1 block receives oral GS₂—As—Se, 200 mg P.O. BID. The patient's walking distance improves to 2 blocks without pain.

### EXAMPLE XLV

A 45-year-old with severe concentric hypertrophy and repeated episodes of shortness of breath is given oral sulforaphane, 40 mg P.O. QID, for 3 months. The patients symptoms of shortness of breath decrease.

### EXAMPLE XLVI

A 60-year-old with a 60-pack/year smoking history is treated with inhaled GSNO (2 cc of 1 millimolar solution Q 6 hrs) with decreased respiratory distress as evidenced by a decrease in respirator support. The patient is then begun on 4,4′-dipyridyl disulfide, 1 nmol/kg/day and the patient is extubated.

### EXAMPLE XLVII

A patient with a cardiomopathy is treated with oral allopurinol-maleimide congener, 300 mg P.O. QD and cardiac pump function increases. Cardiomopathy is associated with a protein with many thiols. Only one thiol is targeted that activates the protein.

### EXAMPLE XLVIII

It is known that NMDS receptors in brain are coupled to nitric oxide synthase (NOS) through a protein denoted PSD95 by virtue of protein-protein interactions. Decreasing these interactions, decreases NOS activity. Decreasing interactions with PSD95 decreases NOS activity. High NOS activity contributes to damage in stroke. A patient with stroke is given inhaled H₂S (10 ppm) to modulate these interactions.

### EXAMPLE XLIX

A 37-year-old with glaucoma receives topical drops, including 10 nanomolar hydrogen peroxide BID. The pressure in the eye decreases.

### EXAMPLE L

A 40-year-old white female with severe sepsis and a blood pressure of 70 mm Hg is begun on an IV infusion of 10 nmol/kg/min of hydrogen peroxide. The blood pressure increases from 60 systolic to 80 systolic.

### EXAMPLE LI

A patient with severe pulmonary fibrosis is begun on inhaled GS₂—As—Se (2 cc 1 millimolar solution inhaled QID) with improvement in PO₂ from 60 at rest to 80.

### EXAMPLE LII

A 40-year-old white female with acute atrial fibrillation post-op is begun on intravenous 4,4′-dipypyridyl disulfide 10 nmol/kg/min and the arrhythmia stops.

### EXAMPLE LIII

A 28-year-old with pulmonary embolism is begun on IV sulforafane 10 nmol/kg/min and a hypoxemia-relieving effect is noted. The PO₂ improves from 70 to 85.

### EXAMPLE LIV

A 40-year-old with ITP receives 10 nmol/kg/min of IV sulforafane with an increase in platelet count 50,000 to 150,000.

### EXAMPLE LV

A 60-year old with diabetes and peripheral vascular disease develops pain on walking one block. The patient is begun on selenite, 10 mg/day, P.O. and within 2 two weeks is walking two blocks.

### EXAMPLE LVI

A 70-year old white make with concentrated ventricular hypertrophy experiences shortness of breath. The patient is given arsenic trioxide, 10 mg P.O. QD, with relief of symptoms.

### EXAMPLE LVII AND LVIII

A 60-year-old with a hematocrit of 25 has a bone marrow biopsy showing a decrease in errythroid lineage. The patient is begun on intravenous 4,4′-dipyridyl disulfide, 40 mg QID for 2 days, with an increase in hematocrit from 25 to 30.

### EXAMPLE LIX

A 40-year-old with diaphragmatic failure receives intravenous 4-hydroxybenzylidene acetone at 1 nmol/kg/min for two days. The patient's respiratory symptoms improved, evidenced by the ability to breathe more easily and the decrease in inspiratory muscle use.

### EXAMPLE LX

A 60-year old white male with recurrent restenosis one month following angioplasty receives oral 4,4′-dipyridyl disulfide, 40 mg P.O. QID, following angioplasty. The patient does not experience restenosis.

### EXAMPLE LXI

A 30-year-old with Hodgkin's disease receives intravenous hydrogen peroxide at 10 nmol/kg/min for 1 hour during radiation therapy. The patient's chest mass decreases in size.

### EXAMPLE LXII

A 27-year-old white male with testicular cancer receives 40 mg P.O. QID of sulforaphane in combination with platinum therapy and the tumor size decreases.

### EXAMPLE LXIII

A 70-year-old with prostate cancer receives 40 mg P.O. QID of 2,2′-dipyridyl disulfide with shrinkage in the size of the tumor. The patient's PSA falls in half.

### EXAMPLE LXIV

A 33-year old black female with recurrent sickle cell crisis and acute chest syndrome is treated with 200 mg P.O. BID of GS₂—As—Se. Her PO₂ improves from 70 to 80 and the frequence and severity of pain crises is decreased by 50%.

Variations of the above will be obvious to those skilled in the art. Thus, the scope of the invention is determined by the claims.

What is claimed is:

1. A method for prophylaxis or treatment of a patient with a disease associated with a protein having a cysteine residue that is modified by a thiol reactive agent either to modulate its function or to inhibit or promote its function, or at risk therefor, comprising administering to said patient a therapeutically effective amount of said thiol reactive agent, with the proviso that the thiol reactive agent is not NO or an NO donor.

2. The method of claim 1 where the disease is an inflammatory condition which is not asthma.

3. The method of claim 1 where the disease is a condition characterized by pathological proliferation.

4. The method of claim 1, wherein the receptor is selected from the group consisting of:
(i) a serotonin receptor;
(ii) an adrenergic receptor;
(iii) a blood cell membrane receptor
(iv) a μ-opioid receptor; and
(v) a G-protein coupled receptor;
(vi) a cyclin; and
(vii) a transcription factor.

5. The method of claim 1, wherein the receptor is a protein.

6. The method of claim 5, wherein the protein is selected from the group consisting of:
(i) a G-protein;
(ii) a metabolic protein;
(iii) a structural or adaptor protein;
(iv) a membrane protein;
(v) an intracellular protein;
(vi) a phosphatase;
(vii) a cysteine protease and wherein the treatment would affect an allosteric cysteine, but not other cysteine; and
(viii) a respiratory protein.

7. The method of claim 1, wherein the receptor is in a cell selected from the group consisting of:
(i) an endothelial cell;
(ii) a cardiac cell;
(iii) a fibroblast;
(iv) an epithelial cell;
(v) a nerve cell;
(vi) a neutrophil;
(vii) a leukocyte;
(viii) a platelet and is not an adenosine diphophate receptor;
(ix) a bone marrow cell;
(x) a skeletal muscle cell; and
(xi) a stem cell or a stem cell lineage related cell.
